(19) United States
(12) Patent Application Publication
(10) Pub. No.: US 2009/0060871 A1
(43) Pub. Date: Mar. 5, 2009

(54) ADMINISTRATION OF AGONIST-ANTAGONIST IN OPIOID-DEPENDENT PATIENTS

(76) Inventors: Michael Victor VORONKOV, Pennington, NJ (US); Efim David NEZHINSKY, Moscow (RU); Levon Gevorkovich ISAKULYAN, Moscow (RU); Daria Alexandrovna OCHERET, Moscow (RU)

Correspondence Address: HOUSTON ELISEEVA, 4 MILITIA DRIVE, SUITE 4, LEXINGTON, MA, 02421, US

(21) Appl. No.: 12/257,457

(22) Filed: Oct. 24, 2008

Related U.S. Application Data
(63) Continuation of application No. PCT/US2007/067101, filed on Apr. 20, 2007.
(60) Provisional application No. 60/825,785, filed on Sep. 15, 2006.
(60) Provisional application No. 60/825,792, filed on Sep. 15, 2006.
(60) Provisional application No. 60/825,799, filed on Sep. 15, 2006.
(60) Provisional application No. 60/890,412, filed on Feb. 16, 2007.

(30) Foreign Application Priority Data
Apr. 25, 2006 (RU) 2006113790
May 4, 2006 (RU) 2006115061

(51) Int. Cl. A61K 31/485; A61K 31/439; A61P 31/12; A61K 31/52; A61K 31/708; A61K 31/513; A61K 31/7072; A61K 31/675; A61K 31/7068; A61K 31/538; A61K 31/551; A61K 31/341; A61K 31/4418; A61K 31/496; A61K 38/21

(52) U.S. Cl. 424/85.4; 514/282; 514/295; 514/263.4; 514/45; 514/274; 514/50; 514/81; 514/49; 514/230.5; 514/220; 514/471; 514/357; 514/253.09

(57) ABSTRACT

A method for reducing psychological opioid dependence includes selecting a patient addicted to an opioid and administering to the patient an opioid agonist-antagonist for at least 14 days, thereby reducing craving for the opioid in the patient. An opioid agonist-antagonist is administered in a patient needing an average adherence to a treatment protocol of at least 95 percent. A non-injectable formulation containing an agonist-antagonist also is disclosed.

### RELATED APPLICATIONS

This application is a Continuation of PCT application serial number PCT/US2007/067101, filed on Apr. 20, 2007, which in turn claims priority to Russian Patent Application Nos. RU 2006113790, filed on Apr. 25, 2006, and RU 2006115061, filed on May 4, 2006, all of which are incorporated herein by reference in their entirety. PCT/US2007/067101 also claims the benefit under 35 USC 119(e) of the following U.S. Provisional Application Nos. which are incorporated herein by reference in their entirety: 60/825,785; 60/825,792; and 60/825,799, all filed on Sep. 15, 2006; and 60/890,412 filed on Feb. 16, 2007.

### BACKGROUND OF THE INVENTION

Substance dependency and abuse are known to affect life expectancy and overall quality of life. Many opiate users, for example, also become infected with the human immunodeficiency virus (HIV) or contract other medical conditions.

The treatment of choice in HIV-infected patients is antiretroviral (ARV) medication which can be administered to prevent or delay the onset of full-blown Acquired Immunodeficiency Syndrome (AIDS). ARV therapy also is useful in managing AIDS patients.

Under the threat of AIDS pandemics, the problem of efficient treatment of this decease is most important. Potent ARV combinations have provided opportunities for effectively treating HIV-infected persons and have led to a dramatic decline in HIV mortality. ARV treatment can inhibit viral replication and markedly delay disease progression; however, achieving these goals often requires careful adherence, e.g., of 95% or greater, to regimens that may be complex and/or cause unpleasant side effects.

Active substance and/or alcohol abuse, however, often are associated with poor compliance to ARV regimens. Non-adherence to the ARV regimen can result in reduced treatment efficacy and the need for further diagnosis and redesign of the ARV protocol. In addition, noncompliance with the prescribed regimen increases the risk of emerging new, therapy-resistant viral strains. Handling drug-resistant HIV strains not only has a negative impact on the patient's health, well-being, and morale but also increases overall treatment costs, placing excessive demands on available resources.

As known, it is difficult to break substance dependency and abuse in both HIV-positive as well as HIV-negative populations.

Often, opioid dependency is addressed through counseling and support group participation. Many substance users also undergo substitution therapy in controlled clinical programs.

During substitution therapy, also referred to as “agonist pharmacotherapy”, “agonist replacement therapy”, or “agonist-assisted therapy”, users receive opioid receptor agonists such as methadone, levo alpha acetyl methadole (LAAM), buprenorphine, or dehydrocodein. Therapy using the opioid antagonist naltrexone also has been proposed. Effects of kappa opioid agonists on cocaine, alcohol, PCP, etc self-administration in primates also have been reported.

Heroin dependency has been addressed using buprenorphine, a partial opioid agonist, in combination with naloxone, an opioid antagonist. Formulations that combine pentazocine with naltrexone have been reported for managing moderate to severe pain. An oral combination of the agonist hydrocodone with naltrexone has been proposed for treating oral abuse of an oral opioid formulation.

While physical symptoms associated with methadone withdrawal are less severe than those experienced with heroin withdrawal, methadone is itself addictive and its effectiveness and safety in managing opioid addiction have been questioned. For example, substitution therapy has been reported to cause euphoric effects, sedative action, breathing depression, constipation and to have a high risk of lethal overdose.

In some cases substitution therapy increases the recreational consumption of alcohol, cocaine and other drugs.

In patients treated for HIV, tuberculosis or for other medical conditions, substitution therapy also can have unfavorable interactions with medication regimens. Methadone, for instance, can have unfavorable interactions with antiretroviral (ARV) medications. Such interactions can precipitate narcotic withdrawal by an ARV agent that induces methadone metabolism and may result in resumption of heroin use and/or in non-adherence to ARV medications. Undesirable side effects also can occur in patients receiving treatment for tuberculosis.

Naltrexone appears to have met with patient reluctance and poor compliance and may be accompanied by liver toxicity. Its administration often requires seven (7) days of abstinence which, for heavy heroin users, is possible only on in-patient basis, resulting in higher costs and poor compliance from the patient.

Combining an agonist compound and an antagonist compound requires balancing and fine-tuning administration of two different drugs, each having its own mechanism of action, metabolism and bioavailability issues, as well as its individual undesirable side effects. The danger of adverse reactions is further increased in patients who also receive treatment for other conditions, such as hepatitis, tuberculosis, herpes or HIV infections. There is a higher risk of abuse, as components can be physically separated.

A need continues to exist, therefore, for managing opioid dependency and in particular for addressing the psychological component of dependency. In this context, a need exists for improved formulations, in particular formulations for non-injectable delivery. A need also exists for improving compliance to ARV or other treatment regimens in patients suffering from opioid-dependency.

### SUMMARY OF THE INVENTION

The invention generally relates to the use of agonist-antagonists such as, for example, nalbuphine.

In one aspect the invention relates to managing opioid addiction. For example, a method for stabilizing inconsistencies associated with opioid dependence includes selecting a patient addicted to an opioid and administering to the patient an opioid agonist-antagonist thereby stabilizing said inconsistencies. The opioid agonist-antagonist preferably is administered for a period of at least 14 days or longer. In one aspect of the invention the opioid agonist-antagonist is nalbuphine.

In another aspect, the invention relates to managing or treating patients suffering from an infection such as HIV, more particularly to assisting such patients to adhere to a treatment protocol.

For instance, a method for maintaining an opioid-dependent patient in compliance with a treatment protocol, for instance an ARV regimen such as administered in HIV-infected individuals includes selecting a patient needing an average adherence to the treatment protocol of at least 95 percent and administering to the patient a treatment component, e.g., ARV medication, and an agonist-antagonist such as nalbuphine. Preferably, the agonist-antagonist is administered for at least one month and at the end this period the patient's average adherence to the treatment regimen is no less than 95 percent.

In another example, a method for maintaining an HIV-infected, opioid-dependent patient on an ARV regimen comprises administering to the patient an ARV component in combination with an opioid receptor agonist-antagonist, wherein the opioid receptor agonist antagonist is administered for no less than a month and the patient's average adherence to the ARV regimen is at least 95 percent.

In yet another example, a method for treating an HIV-infected patient includes selecting an opioid-dependent patient having an average adherence to an ARV regimen that is less that 95 percent; and administering to the patient ARV medication in combination with nalbuphine.

In a further example, a method for maintaining an average compliance to an ARV regiment of at least 95 percent in an opioid-dependent patient comprises administering to the patient ARV medication in combination with an opioid receptor agonist-antagonist

In a further aspect, the invention relates to a non-injectable composition suitable for managing opioid addiction. An active component including an agonist-antagonist can be formulated with an inactive component in the form of a tablet, capsule, syrup, suppository, inhalable powder, inhalable aerosol, sublingual spray, intranasal spray, intranasal aerosol or another non-injectable formulation. More than one agonist-antagonist(s) can be present in the formulation. In addition to the agonist-antagonist(s), the composition disclosed herein can include one or more agonist(s) and/or one or more antagonist(s). In a preferred example, the formulation is a tablet or caplet which includes pharmaceutically acceptable nalbuphine salt or its derivative and a release modifying component. Other ingredients also can be present.

The invention has many advantages. It can be practiced in both males and females and presents no restriction with respect to age.

The invention results in overall improvements in quality of life and is particularly well suited in addressing the psychological component of opioid addiction, stabilizing psychological and behavioral inconsistencies related to substance abuse. Patients receiving agonist-antagonists are found to stay away from illicit drugs during agonist-antagonist therapy, show a reduction of criminal and asocial activities and make progress in social life. Suicide attempts, suicide deaths and overdose events are reduced. Once on an agonist-antagonist treatment regimen, risky injection behavior related to blood-born infection and re-infections decreases. In those receiving ARV treatment for managing HIV or other infections, such as herpes, tuberculosis or hepatitis B and C, compliance with the ARV protocol is increased, thereby reducing adverse effects on the patient's health and/or life threatening consequences.

While binding to κ-receptors, administration with agonist-antagonists does not produce the euphoric effects due to antagonistic binding to μ-receptors. It is found that agonist-antagonists block not only opiates but decrease alcohol consumption and instances of poly-narcotic abuse.

Using an agonist-antagonist which does not have as strong sedative effect, such as, for instance, nalbuphine, minimizes impairment of cognitive processes and/or respiration and protects patients from substances abuse, thus enhancing the patient's quality of life.

In contrast to substitution therapy, the antagonist component present in agonist-antagonists, minimizes and, in most cases, eliminates concurrent consumption of illicit drugs during the course of therapy, thus reducing or minimizing the possibility of an overdose. Thus the method of the invention provides a safer and more effective treatment than is obtained using substitution therapy.

In contrast to antagonist therapy (e.g. naltrexone) the agonist component present in agonist-antagonist alleviates (eliminates) cravings for an illicit drug preventing a patient from attempting what is known as “breaching” antagonist (e.g. naltrexone) blockade with higher doses of an illicit drug. Thus threat of death from the related overdose is greatly diminished. Adherence to the proposed treatment is much higher than with antagonist therapy. Furthermore, in contrast to naltrexone, an agonist-antagonist such as nalbuphine requires only 3 day of abstinence.

A further advantage associated with the agonist-antagonist nalbuphine may be related to its distinct opiate receptor signature: it is an antagonist of μ- (mu) receptors, an agonist of κ- (kappa) receptors and it apparently has little or no affinity toward other receptors. While, as described above, therapy with μ-receptor antagonists (e.g. Naltrexone) is being practiced and while there are some reports on efficacy of κ-receptor agonists in primates to modulate substance abuse, nalbuphine can engage both μ (mu) and κ (kappa) mechanisms to mediate psychotomimetic effects of illicit drugs. Since it appears to have no significant interactions with other opiate receptors, nalbuphine can be used as a research tool, providing a simple and clean clinical profile and rendering interpretation of data straightforward and unambiguous.

The invention can provide high efficiency in treating HIV-infection due to the high patient compliance to the ARV therapy. Emergence of resistant strains of the virus is minimized, resulting in better patient care and reduced treatment costs. Average adherence to ARV protocols obtained practicing the invention compares favorably with that observed with methadone, while some of the undesirable drug interactions between methadone and ARV medication are reduced or eliminated.

In an opioid-dependent patient, the non-injectable formulation disclosed herein is easy to use and breaks the association (mental, psychological, behavioral, etc.) of the agonist-antagonist therapy with injectable narcotics such as heroin. Thus, efficacy of treatment is increased and the likelihood of relapse reduced. The non-injectable formulation designed for oral administration resists destruction by digestive enzymes and has controlled or prolonged release properties.

### DETAILED DESCRIPTION OF THE PREFERRED EMBODIMENTS

The above and other features of the invention including various novel details of construction and combinations of parts, and other advantages, will now be more particularly described and pointed out in the claims. It will be understood that the particular method and/or device embodying the invention are shown by way of illustration and not as a limitation of the invention. The principles and features of this invention may be employed in various and numerous embodiments without departing from the scope of the invention.

The invention is practiced with human subjects, and is applicable to both males and females, without age restrictions. The invention also can be practiced in non-human patients, such as, for instance, laboratory animals.

In one aspect, the invention relates to managing opioid-dependency.

As used herein, the term “opioid” refers to any endogenous or exogenous agent that binds to opioid receptors, found principally in the central nervous system and gastrointestinal tract. There are four broad classes of opioids: endogenous opioid peptides, produced in the body; opium alkaloids, such as morphine (the prototypical opioid) and codeine; semi-synthetic opioids such as heroin and oxycodone; and fully synthetic opioids such as pethidine and methadone that have structures unrelated to the opium alkaloids. The term “opiate” often is used to refer to the natural opium alkaloids and the semi-synthetics derived from them.

In a preferred example, the opioid is heroin. Dependency on other opioids, such as, codeine, codeine phosphate, tramadol, home-made acetyl-morphine or combinations thereof also can be managed.

Patients that can be selected also include those who take one or more opioid compound in combination with other substances known to cause dependency. Examples include patients who, in addition to being dependent on opioids, abuse alcohol, habitually smoke marijuana, abuse amphetamines, cocaine, crack cocaine, gammahydroxybutyrate (GBH), and so forth.

Dependence or abuse of an opioid can be established by considering several factors, including: the capacity of the drug to produce the kind of physical dependence in which drug withdrawal causes sufficient distress to bring about drug-seeking behavior; the ability to suppress withdrawal symptoms caused by withdrawal from other agents; the degree to which it induces euphoria similar to that produced by morphine and other opioids; the patterns of toxicity that occur when the drug is dosed above its normal therapeutic range; and physical characteristics of the drugs such as water solubility.

At least three basic patterns of opioid use and dependence have been identified. The first involves individuals whose drug use begins in the context of medical treatment and who obtain their initial supplies through, e.g., physicians. Another pattern begins with experimental or “recreational” drug use and progresses to more intensive use. A third pattern involves users who begin in one or another of the preceding ways but later switch to oral opioids such as methadone, obtained from organized addiction treatment programs.

Opioid-dependence often is accompanied by tolerance towards the opioid. Tolerance refers to the need to increase the dose of opioid over a period of time in order to achieve the same level of analgesia or euphoria, or the observation that repeated administration of the same dose results in decreased analgesia, euphoria, or other opioid effects. It has been found that a remarkable degree of tolerance develops to the respiratory depressant, analgesic, sedative, emetic and euphorigenic effects of opioids. However, the rate at which this tolerance may develop in either an addict or in a patient requiring treatment of pain depends on the pattern of use. If the opioid is used frequently, it may be necessary to increase the dose. Tolerance does not develop equally or at the same rate to all the effects of opioids, and even users who are highly tolerant to respiratory depressant effects continue to exhibit meiosis and constipation. Tolerance to opioids largely disappears when the withdrawal syndrome has been completed.

Opioid dependency can be determined by criteria established by the medical community. Approaches that can be used to establish opioid dependence in a patient include but are not limited to interviews conducted by qualified personnel, overall clinical examinations, needle marks, withdrawal symptoms, clinical testing, e.g., urine or blood tests, statements from family or friends, and so forth.

As used herein, the term “opioid-dependence” refers to both physical and psychological dependence.

Physical dependence may develop upon repeated administrations or extended use of opioids. Physical dependence is gradually manifested after stopping opioid use or is precipitously manifested (e.g., within 20 minutes) after administration of a narcotic antagonist (referred to “precipitated withdrawal”). Depending upon the drug to which dependence has been established and the duration of use and dose, symptoms of withdrawal vary in number and kind, duration and severity. The most common symptoms of the withdrawal syndrome include anorexia, weight loss, pupillary dilation, chills alternating with excessive sweating, abdominal cramps, nausea, vomiting, muscle spasms, hyperirritability, lachrymation, rhinorrhea, goose flesh and increased heart rate. Abstinence syndrome typically begins to occur 24-48 hours after the last dose, and the syndrome reaches its maximum intensity about the third day and may not begin to decrease until the third week.

Psychological dependence (i.e., addiction) to opioids is characterized by drug-seeking behavior directed toward achieving euphoria and escape from, e.g., psychosocioeconomic pressures. An addict will continue to administer opioids for non-medicinal purposes often in the face of self-harm.

In one embodiment, the invention is directed to a method for stabilizing inconsistencies associated with opioid dependence. As used herein, the term “inconsistency” refers in to fluctuations in behavior patterns and psychological responses. Examples of inconsistencies can be seen in poor or erratic work attendance, criminal activities, non-compliance with medical treatment regimens, lapses in parental duties and so forth.

To manage opioid usage or addiction, and in particular to obtain stabilization in inconsistencies such as described above, the patient is administered an opioid receptor agonist-antagonist. More than one opioid agonist-antagonist can be used. In further aspects of the invention, the opioid agonist-antagonist component is administered in combination with one or more opioid agonist(s). The opioid agonist-antagonist component also can be administered in combination with one or more opioid antagonist(s). As used herein, the term “opioid agonist-antagonist component” refers to one or more agonist-antagonist(s).

In a preferred example, the agonist-antagonist is nalbuphine or 17-(cyclobutylmethyl)-4,5α-epoxymorphinane-3,6α-14-triol. Chemically, nalbuphine has the structural formula shown below:

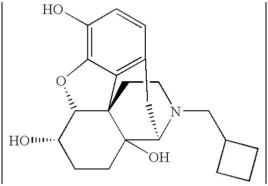

As used herein, the term “nalbuphine” includes nalbuphine, nalbuphine hydrochloride, nalbuphine esters, pharmaceutically acceptable salts, complexes and derivatives of nalbuphine or nalbuphine esters, as well as to other suitable nalbuphine derivatives and salts of thereof.

Nalbuphine chloride or 17-(cyclobutylmethyl)-4,5α-epoxymorphinan-3,6α-14-triol hydrochloride is available as a white or nearly white crystalline powder. Water-soluble, it has a chemical formula C21H27NO4·HCl, a molecular weight of 393.91 g/mol. Its structure is shown below:

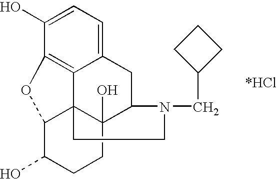

Currently, nalbuphine hydrochloride is indicated for relief of moderate to severe pain, as a supplement to balanced anesthesia, for pre-operative and post-operative analgesia and for obstetrical analgesia during labor and delivery. It is available commercially from Endo Pharmaceuticals, Chadds Ford, Pa. under the registered trademark NUBAIN® in injectable sterile solutions for intramuscular (IM), subcutaneous (SC) or intravenous (IV) administration.

A potent analgesic, nalbuphine hydrochloride has an analgesic potency which is essentially equivalent to that of morphine on a milligram basis. Its onset of action occurs in within 2-3 minutes after IV administration and in less than 15 minutes after SC or IM administration. Its plasma half life is about five hours. Clinical results place the duration of action of nalbuphine hydrochloride within the range of from about 3 hours to about six hours.

Nalbuphine esters are described, for instance, in U.S. Pat. No. 5,750,534, to Yoa-Pu et al., issued on May 12, 1998, the teachings of which are incorporated herein by reference in their entirety. The chemical formula for a nalbuphine monoester is shown below:

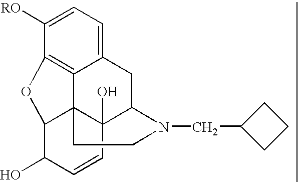

Preferred nalbuphine monoesters include, but are not limited to, nalbuphine propionate, nalbuphine pivalate, nalbuphine enanthate, nalbuphine decanoate, nalbuphine behenate, nalbuphine erucicate, nalbuphine arachidate, and nalbuphine benzoate.

Nalbuphine polyesters also can be used. As described in U.S. Pat. No. 6,225,321 issued to Hu et al. on May 1, 2001, the teachings of which are incorporated herein by reference in their entirety, nalbuphine polyesters have the general formula shown below:

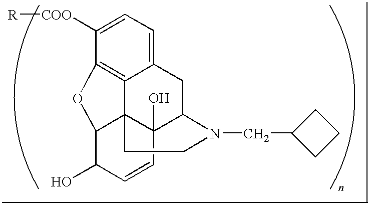

where n is an integer, e.g., from 2 to 4 and R can be a saturated, unsaturated, substituted or unsubstituted aliphatic or aromatic group having, e.g., 1 to 40 carbon atoms.

Suitable derivatives of nalbuphine polyester are described, for instance in U.S. Pat. No. 6,225,321 B1, issued to Hu et al. on May 1, 2001, the teachings of which are incorporated herein by reference in their entirety.

Pharmaceutically, nalbuphine is classified as a synthetic opioid agonist-antagonist analgesic of the phenanthrene series and is related to the opioid antagonist naloxone and the opioid analgesic oxymorphone.

As used herein, the term opioid agonist refers to an active agent which binds to a receptor and triggers a cell response. As used herein, the term opioid antagonist refers to an adverse agent. Opioid antagonists also are believed to bind to a receptor but generally do not activate the receptor; rather opioid antagonists block the receptor from activation by agonists.

In 1973 K. Pert and S. Snyder discovered “opioid receptors” to bind exogenous morphine. Later a number of subtypes of these receptors were discovered: μ- (mu), κ- (kappa), σ- (sigma) and δ- (delta) localized in CNS and responsible for sensitivity to pain. The μ, κ, and δ opioid receptors have been reclassified by an International Union of Pharmacology subcommittee as OP 1 (delta), OP2 (kappa), and OP3 (mu).

It is believed that nalbuphine acts on specific opiate-receptor subtypes: it is a potent μ-antagonist with less dysphoric effects, and its agonistic effects at κ1- and κ3-receptors provide analgesia. Actions of nalbuphine at the kappa-receptors are believed to produce alterations in the perception of pain as well as the emotional response to pain, possibly by altering the release of neurotransmitters from afferent nerves sensitive to painful stimuli. As an adjunct to anesthesia, nalbuphine protects against the hemodynamic responses to stress produced by surgery. As a function of antagonism at the μ-receptor, nalbuphine can induce opiate withdrawal if administered to opiate-dependent patients.

In one example, injectable nalbuphine is administered at 2 cc twice daily. Other doses can be employed, as known in the medical arts.

To effect dissociation between agonist-antagonist administration and the use of needles in illicit drug use, it is preferred that the agonist-antagonist is administered in a non-injectable formulation.

The non-injectable formulation can be provided for oral administration, e.g., sublingual tablet, prolonged form of tablet, caplet, capsule, gel caplet, as a syrup, solution and so forth.

In other examples, the non-injectable formulation is provided for transdermal or subdermal implant and/or patch. Other non-injectable formulations include suppositories, inhalable aerosols or powders, drops, sprays, aerosols or other preparations for nasal or ocular delivery, and so forth.

The non-injectable formulations of the invention can be tailored for immediate bioavailability or for sustained release. In further aspects of the invention, more than one agonist-antagonist can be present in the formulation.

Preferably, the formulation includes an effective amount of the agonist-antagonist. In the case of nalbuphine, the non-injectable formulation can include an amount that, when administered, would result in an effect similar and preferably equivalent to an effective dose of injectable nalbuphine, e.g., 2 cc.

In addition to the agonist-antagonist component, the non-injectable formulation can include additional ingredients.

For example, the formulation can further include additional active ingredients such as, for instance, pain killers, antibacterial, antiviral or antifungal agents, vitamins, immune system fortifiers, homeopathic agents, antihypertensive medication, nootropics, any combinations thereof, and so forth.

Inactive ingredients also can be included. Such inactive ingredients can be used for bulk, drug release properties, as a carrier, for facilitating digestion, and for other purposes, as known in the art.

Some inactive agents that can be employed include, but are not limited to ammonio-methacrylate copolymers, NF, fumaric acid, NF, povidone, USP, sodium lauryl sulfate, NF, sugar starch, NF, and talc, USP, gelatin, titanium dioxide, lactose, hydroxypropyl methylcellulose, colloidal silicon dioxide, stearic acid, lactic acid, citric acid, vitamin E, EDTA, butylated hydroxyanisole, propylparaben, methylparaben, sodium benzoate, potassium benzoate, benzalkonium chloride, benzoic acid, sorbic acid, PEG 400, carrageenan products (such as Viscarin 328, Gelcarin 812, and Seaspen) and others.

In a specific, non-limiting example, the non-injectable formulation is a capsule containing 5-150 mg (e.g. either 10, 60, 90, or 120 mg) of nalbuphine, and one or more of the inactive ingredients such as ammonio-methacrylate copolymers, NF, fumaric acid, NF, povidone, USP, sodium lauryl sulfate, NF, sugar starch spheres, NF, and talc, USP, (and their suitable analogues). The capsule shell can contain ink, gelatin, titanium dioxide, (and their suitable analogues).

In another specific, non-limiting example, the non-injectable formulation is a tablet including 5-150 mg (e.g. either 10, 60, 90, or 120 mg) of nalbuphine. Inactive ingredients are, for instance, lactose, hydroxypropyl methylcellulose, colloidal silicon dioxide, and/or stearic acid. Known analogues of the inactive components also can be used.

In further examples, nalbuphine is administered orally using a formulation such as described, for instance in U.S. Pat. No. 6,703,398 B2, issued on Mar. 9, 2004 to Hu et al, the contents of which are incorporated herein by reference in their entirety.

When administered orally, nalbuphine chloride is partially destroyed by enzymes present in the digestive system. A preferred nalbuphine chloride formulation, capable of withstanding enzyme activity in the digestive tract and useful in the prophylaxis and/or suppression of narcotic addiction caused by opioid narcotic substances, is described below.

The formulation is in solid form and can be manufactured as coated or uncoated tablets or caplets.

In addition to nalbuphine chloride, the formulation includes other ingredients. Resistance to the action of digestive enzymes and drug release modification properties are some of the features used when selecting some or all of these ingredients. Ingredients that have a tendency to swell when placed in an aqueous medium also are preferred.

In one example, the formulation includes nalbuphine, a release modifying component and optional additives.

The release-modifying component can include one or more materials. Preferably, the one or more materials prolong the release of nalbuphine from the formulation.

Examples of release-modifying materials include carbomers, carboxymethylcellulose, as well as any combination thereof.

Carbomers, e.g., Carbopole resins are compounds which are carboxyacrylic or carboxyvinyl polymers, such as described in USA pharmacopeia monographs, in British and other European and Japanese and other pharmacopoeias.

Hydroxypropylmethylcellulose is propyleneglycol and methylcellulose ether, as described in USA pharmacopoeias and also in British and other European pharmacopeia.

Biodegradable polymers and other release-modifying agents suitable for oral administration also can be utilized.

Additives such as, for example, lactose, microcrystalline cellulose, colloidal silica, lubricants, acid stabilizers, disintegrants and many others also can be included.

An example of the formulation in tablet form includes by weight percent:
Nalbuphine hydrochloride: 10-55
A release modifying component: 10-35
Colloid silicon oxide e.g., Aerosil: 0.2-3
Microcrystalline cellulose: 5-20
Povidone: 1-5%
Lubricants: 0.3-5%
Lactose: the rest.

Microcrystal cellulose (MCC) is a multifunctional compound, providing necessary strength properties of the dosage form.

Colloid silicon oxide, for instance, Aerosil, is a material that can improve allocation of components in process mixture mass. Aerosil granular forms can be used for additional improvement of rheological properties of the tablet mass.

Examples of lubricants include magnesium stearate, calcium stearate, talcum, their mixtures and others, as known in the art. It is preferably to use magnesium stearate in the quantity of 0.2-1.5% and talcum in the quantity of 0.8-3.0%.

Povidone is a synthetic water-soluble polymer made from the monomer N-vinyl pyrrolidone.

Lactose is a neutral filler, providing optimal rheological properties of the granulated material and tablet mass in the manufacture of the tablet. Lactose having particle size of 70-200 microns, are preferred. Also preferred are spherical or nearly spherical lactose particles.

Without wishing to be held by a specific mechanism of action, it is believed that compounds employed in the present formulation can form a protective matrix around nalbuphine chloride and modify its release kinetics from the formulation.

The tablet or caplet described above can be formulated at a desired dosage, for example it can contain 10-150 mg of nalbuphine chloride.

In another example, the formulation includes:
The preferred composition is as follows:
Nalbuphine—16-33%
Carbopole—25-40%
Aerosil—0.5-1%
Microcrystallic cellulose—9-25%
Magnesium silicate—0.5-2.5%
Magnesium stearate—0.5-1%
Lactose—the remaining balance
The tablet is coated with Opadry YS-1-7027 (white) and ACRYL-EZE (white) with antifoaming emulsion in the following ratio:
Opadry YS-1-7027 (white)—16-21.5%
ACRYL-EZE (white)—78-83.5%
Antifoaming emulsion—the remaining balance.

The stated proportion of the ingredients is optimal, was determined in experiments, and provides necessary quality of the composition and validity period more than 2 years.

The non-injectable formulations disclosed herein can be prepared by combining one or more agonist-antagonists with any other active or inactive ingredients. The process is not limited to any particular order of adding ingredients. One or more ingredients can be added simultaneously and sequential additions also can be carried out. Laboratory, pilot plant and commercial operations can be employed. Mixing, spray drying, emulsifying, purifying, compounding, and many other additional steps known in the fields of drug synthesis and manufacture also can be used to produce the non-injectable formulation.

The formulation can be provided in a kit. The kit can include the formulation arranged according to an administration schedule. Thus tablets or caplets can be provided in blisters or pouches arranged on one or more sheets, with rows and columns labeled to facilitate tracking a desired administration schedule.

The kit can further comprise an applicator, for example, a pipette, a dropper, a spray, an inhaler, a nebulizer, enema equipment and others known in the art.

Instructional materials describing the antibody component, methods for using the applicator, possible adverse reactions, and other information also can be included in the kit.

Non-injectable formulations such as those described herein can be administered for at least two weeks (14 days), preferably for about one month or longer.

Schedules for administering non-injectable formulations can vary, for example, from multiple times a day to once daily. Other administration schedules can be used. Controlled release formulations, for instance, can be administered less frequently than once daily and transdermal devices can be designed for replacement at weekly, bi-weekly or longer intervals.

The following criteria for effectiveness of substitution therapy recommended by the World Health Organization (WHO) also can be applied to evaluate the stabilization method disclosed herein. They include: i) reduction of consumption or staying away from illicit drugs for long time; ii) reduction of criminal and asocial activities of the patient; iii) prevention of HIV and other blood-born infection or compliance with the therapy (or any other therapy, such as ARV, tuberculosis, etc.); iv) reduction of suicide attempt, deaths from overdose.

Results so far indicate high effectiveness when patients with psychological and behavioral inconsistencies related to substance abuse are treated with the stabilization therapy disclosed herein. Using an agonist-antagonist such as nalbuphine results in all or some of the following: A) staying away from illicit drugs for time of treatment; B) reduction of criminal and asocial activities of the patient; C) progress in social life D) decrease of risky injection behavior related to blood-born infection and re-infections; and E) reduction of suicide attempts, suicide death and death from overdose.

Without wishing to be bound by any specific interpretation, it is believed that nalbuphine has a distinct opiate receptor signature: it is an antagonist of μ-receptors and also an agonist of κ-receptors. At the same time, it appears to have little or no affinity toward other receptors. Thus nalbuphine is believed to engage both μ and κ mechanisms to mediate psychotomimetic effects of illicit drugs and to do so without significant interactions with other receptors.

It was further discovered that administration of an opioid agonist-antagonist also increases adherence to a medication protocol. The terms “protocol” and “regimen” are used herein interchangeably and refer to the medication, its dosage, administration frequency, route and so forth, for treating or managing a condition such as, for instance, HIV infection, herpes, tuberculosis, hepatitis C or other medical conditions. In most cases, the treatment regimen is determined and prescribed by medical personnel and preferably is tailored to a particular patient.

In managing an infection such as HIV, for example, it is important that the patient complies with a treatment protocol or regimen. Some HIV-positive patients, for instance those in more advanced stages of HIV, need to comply with an ARV protocol. As used herein, “ARV” includes known ARV agents as well as medicaments being developed now or in the future for managing HIV, tuberculosis, herpes, hepatitis C or other viral infections. Natural compounds that act to inhibit, or reduce replication or other function in viruses, and in particular in retroviruses, also can be used.

ARV agents often are classified by their mechanism of action and include reverse transcriptase inhibitors, protease inhibitors, integrase inhibitors, attachment and fusion inhibitors, antisense drugs, immune modulators and others.

Specific examples of typical antiviral drugs currently employed in managing HIV-positive patients include but are not limited to zidovudine (AZT), didanosine, enteric-coated didanosine, zalcitabine, stavudine (d4T), extended release stavudine, lamivudine (3TC), acabavir, tenofovir, emtricitabine (FTC), nevirapine (NVP), delaviridine, efavirenz, saquinavir, ritonavir, indinavir, nelfinavir, amprenavir, lopinavir, atanazavir, fosamprenavir, tipranavir, enfurvirtide, acyclovir, interferons, ribavirin and others.

Preferably, HIV-infected patients are administered a combination or “cocktail” of ARV drugs. For example, a patient may receive efavirenz, in combination with: (a) 3TC or FTC; and (b) AZT or tenofovir or d4T. Another typical combination regimen includes nevirapine and lamivudine.

Among compounds under development, PA-457, for example, is a maturation inhibitor currently in clinical testing. Its activity does not appear to target an enzyme or a receptor. Rather, PA-457 is believed to inhibit the last step in gag processing in which the viral capsid polyprotein is cleaved, thereby blocking the conversion of the polyprotein into the mature capsid protein (p24). Because these viral particles have a defective core, the virions released consist mainly of non-infectious particles.

Other approaches relate to the APOBEC family of proteins produced by the body that help to protect it against certain viruses and much interest today concerns one of these proteins, APOBEC3G (also called CEM15). Since HIV is able to infect humans only because it has a gene, Vif (viral infectivity factor) that blocks APOBEC3G also is being investigated. It has been reported that Vif acts by keeping APOBEC3G from getting where it is needed, speeding up its destruction, and making it work less efficiently. As more is understood about these mechanisms, one or more of them might be a target for an entirely new kind of anti-HIV drug.

Taking a different route, researchers in Switzerland have treated a few patients with a combination of monoclonal antibodies, after they discontinued antiretroviral treatment. The antibodies seemed to work at least as well as HAART (Highly Active Antiretroviral Therapy) in suppressing HIV, except in one patient, whose virus rapidly returned, probably due to viral resistance to those particular antibodies.

Treatment regimens typically being prescribed can vary depending on the stage of the infection, the nature of the medication used, its pharmaceutical formulation, route of administration and so forth. In preferred aspects of the invention, ARV administration follows protocols known and prescribed by medical professionals for managing HIV-positive patients.

There are two unproven approaches to managing early HIV infection in asymptomatic patients: aggressive and conservative. For people with CD4+ cell counts above 350 and viral load above 30,000 (by bDNA) or 55,000 (by RT-PCR), there are no available data to suggest which approach results in longer survival. Very early, aggressive treatment might lead to longer life. Or it might lead to using up the limited supply of therapies too early in the course of disease. Moreover, it also risks early exposure to possible long-term side effects associated with therapies. As a result many experts would delay starting therapy and continue to check CD4+ cell counts and viral load. On the other hand, the risk of disease progression over the next three years is somewhat high (over 30%) in people who meet this definition and other experts prefer to start treatment without further delay.

In more advanced stages, one common therapy presently prescribed includes zidovudine (AZT) (300 mg)+lamivudine (3TC) (150)+nevirapine (NVP) (200 mg) orally twice a day, or as individually determined.

In one example, all ARV medicaments are administered at the same time. Administration can be daily or can be at higher or lower frequency. In other examples two, three or more ARV drugs are administered on individualized schedules, for instance at times staggered with respect to one another.

Pharmaceutical formulations designed for oral administration are preferred but ARV medicaments can be formulated for other routes of administration, e.g., injection.

In some cases, two, three or more ARV agents are combined in a single pharmaceutical formulation, for instance a single pill. Combivir®, for example, combines lamivudine and zidovudine, while Trizivir includes AZT, 3TC and abacavir. A pill combining lamivudine, zidovudine and nevirapine has recently received tentative FDA approval.

As used herein, the terms “treatment component” or “ARV component” refer to one or more agents, medicaments or drugs administered to the patient in order to manage or treat their condition, e.g., HIV infection, herpes, hepatitis and so forth.

Compliance with a treatment regimen and in particular to an ARV regimen is determined using an “average adherence” value calculated using the formula:

(A−B)/A 100%

where A is the number of tablets or gel capsules required by the protocol to be administered to a patient over four weeks; and B is the number of tablets or gel capsules prescribed but actually not taken by a patient over the same period of time.

Average adherence values can similarly be established for patients taking non-tablet or non-gel capsule medication, e.g., injections, inhalation, suppositories, and so forth. As with tablets or gel capsules, the average adherence value will be determined using the number of doses prescribed and those actually taken over a period of time, e.g., four weeks.

Since the exact level of adherence that is necessary to prevent the emergence of drug-resistant virus or to delay disease progression and death remains unknown, near-perfect adherence, e.g., greater than 95%, as recommended by the European AIDS Treatment Group, is considered the goal for HIV-infected patients. In preferred embodiments of the invention, an “average adherence” of at least 95% is preferred. In some cases, prior to carrying out the method of the invention, the patient has an average adherence to a treatment regimen that is less than 95 percent, sometimes less than 90 percent.

To maintain an opioid-dependent patient in compliance with the regimen, for instance by stabilizing psychological or behavior inconsistencies, the patient receives, in addition to a treatment component, e.g., ARV component, nalbuphine or another agonist-antagonist.

Schedules for administering ARV and nalbuphine preferably coincide, but can differ from one another. For instance, ARV medication often is administered twice daily, with nalbuphine administration at the same time. In some cases, ARV can be administered once daily and nalbuphine twice daily. Other administration schedules can be followed. Often nalbuphine is administered by a route that is different from that used to administer an ARV drug or combination or ARV drugs. For instance, the ARV drug or drug combination can be administered orally, in tablet or gel capsule form, while nalbuphine often is administered by IV, SC or IM injection.

The treatment described herein can be accompanied by administration of other drugs or compounds such as antibiotics, antifungal agents, pain killers, vitamins, immune system fortifiers, homeopathic agents, antihypertensive medication, nootropics and so forth.

The invention can be practiced with agonists-antagonists other than nalbuphine and combinations of the agonists-antagonist with one or more agonist(s), antagonist(s), or agonist-antagonist(s) also can be employed. The invention can be practiced with agonists-antagonists other than nalbuphine and combinations of the agonists-antagonist with one or more agonist(s), antagonist(s), or agonist-antagonist(s) also can be employed.

As described above with respect to nalbuphine, the analgesic properties of an agonist-antagonist are due to agonistic binding to κ-receptors, while it does not produce pronounced euphoric effect due to antagonistic binding to μ-receptors. The agonist-antagonist combines a centrally acting (i.e., cross the blood brain barrier) agonists of a κ-opioid receptor and a centrally acting opioid antagonist such that the analgesia achieved by this administration is greater than with administration of either the κ-opioid receptor agonist or the opioid antagonist alone.

Specific examples of agonist-antagonists other than nalbuphine that can be employed include but are not limited to butorphanol, pentazocine and so forth. In one example, the agonist-antagonist is a compound currently used in antidiarrheal medication. The compound has κ- or σ-opioid receptor agonist activity, combined with μ-opioid receptor antagonistic activity.

Other synthetic, semi-synthetic and natural agonist-antagonists with various affinities to different subclasses of opioid receptors also can be used. In the context of the invention agonists-antagonists could be any class of compounds (synthetic, semisynthetic or natural) which bind to or compete for opioid receptors (μ-, κ-, σ-, δ-), for example agonists, antagonists, agonists-antagonists, partial agonists, partial antagonists, and their combinations.

The invention is further illustrated by the following examples, which are not intended to be limiting.

### EXEMPLIFICATION

### A. Clinical Examples

### Example 1A

### C1-P-Rus

M., male, 24 years old, married, has 1 child, employed had a heavy opioid dependency and alcohol abuse. From 2001 M has been registered at the municipal drug treatment clinic.

M started to use psychoactive drugs, including heroin, alcohol, amphetamines, cannabis, phencyclidine and ketamine at the age 14. According to the data of the last HIV antibody test (in 2000), M was HIV-negative. M had chronic hepatitis C virus (HCV) infection since 2000 and did not undergo HCV treatment.

During the period of his drug abuse, M. had multiple attempts to abstain from drugs and alcohol. During the last several month before starting nalbuphine therapy M used heroin, amphetamines, methamphetamine and MDMA.

In April 2006 M. was administered nalbuphine, 2 cc IM twice a day for a period of 48 days. The treatment led to discontinuance of the use of illegal street drugs and alcohol. M's mental state improved, and he reports of profound decrease in craving. No aggravation of opportunistic infections have been observed, neither of HCV. M has found a new job and restarted education in college.

### Example 2A

### C2-P-Rus

D., female, 27 years old, married, has 1 child, employed presented with heavy opioid dependency. At the age 15 D started to use psychoactive drugs, including heroin, ketamine, cocaine, amphetamines, and benzodiazepines. D. reported a psychiatric examination in 1995 when she was diagnosed schizophrenia but did not get specialized psychiatric treatment.

According to the data of the last HIV antibody test (in 2006) D is HIV-negative and has unconfirmed HCV infection.

D. has been twice in drug treatment programs: in 1995 and 2005 with the longest period of remission being 3 weeks. Several times D. had self-managed attempts to stop drug use. D. reported “minor” drug use during pregnancy and breastfeeding. In 2006 D. used heroin, amphetamines, methamphetamine and MDMA.

In April 2006 D. was administered nalbuphine, 2 cc IM twice a day for 52 days that led to discontinuance of the use of illegal street drugs. Her mental state improved, and she denies desire for drug use. D. reports an increase in work capacity and improvement of family relationships.

### Example 3A

### C3-P-Rus

N., female, 26 years old, not married, HIV-positive (symptomatic stage), had heavy opioid dependency. From the age of 16 N. regularly used psychoactive drugs: opiates (heroin, codeine), and amphetamines.

N. was diagnosed HIV-positive in 1999 (way of transmission—injecting drug use). And receives HIV treatment in the Moscow AIDS center. Her co-infections included chronic viral hepatitis C and her opportunistic infection were herpes and CMV.

From 2004 (immune status 157, viral load—2900) N. started ART with Viracept, Zerit, Epivir. Her last laboratory tests (January 2006) showed undetectable viral load and immune status—350. Average adherence to ARVs in January-March 2006 has been 98%.

N. complained of severe chronic pains related to chronic osteomyelitis. To treat chronic pain, N. was administrated non-steroid pain-killers, with low effectiveness.

During the period of her drug use, N. tried to stop drug use many times, but the maximum duration of the remission has never been more than 2 months. She explains failures of her last attempts with the necessity to “relieve pain”.

In April 2006 N. was administered nalbuphine, 2 cc IM twice a day for 30 days that led to both pain relief and discontinuance of the use of heroin and other illicit drugs. Her mental state profoundly improved and the cravings for drugs decreased. Average adherence to ART during April has been 100%. No aggravation of opportunistic infections have been observed, neither of HCV.

### Example 4A

### C1-P-Rus

S., male, 28 years old, divorced, has 2 children (in 2006 deprived in parental rights), unemployed, HIV-infected (symptomatic stage), heavy opioid dependency.

S. started to use psychoactive drugs, including heroin, amphetamines, cannabis, and alcohol at the age of 15 and had multiple craniocerebral traumas. S. never obtained drug treatment or psychiatric treatment.

S. was diagnosed HIV-positive in 2000 (way of transmission—injecting drug use) and has been receiving HIV treatment at the Moscow AIDS center. Co-infections: HCV. Opportunistic infections: CMV, herpes, fungoid infections.

In November 2005, S. started antiretroviral therapy with Viracept and Combivir because of the progression of HIV disease (CD4-37, viral load—270 000).

In January 2006 the immune status increased up to 149, and the viral load was 2 000. In March 2006 Viracept was replaced with Stocrin because of problems with the regimen. Now S. gets Stocrin+Combivir. Average adherence to ARVs in January-March 2006 has been 76%.

S. had many self-maintained attempts to abstain from drugs. During the period of ARV treatment he tried to stop drug use twice; however, the drug use was resumed.

In April 2006 S. was administered Nalbuphine, 2 cc IM twice a day, leading to discontinuance of the use of heroin and ‘street drugs’ (self-made opium and codeine). His mental state improved, and he reports a profound decrease in craving for the drugs. Average adherence to ART during April has been 97%. No aggravation of opportunistic infections have been observed, neither of HCV.

### Example 5A

### C2-P-Rus

V., male, 33 years old, divorced, has 1 child (in 2001 deprived in parental rights), unemployed, is HIV-infected (symptomatic stage) and had a heavy opioid dependency.

At the age of 14, V. started to use psychoactive drugs; opioids followed from the age of 16. Besides opioids (heroin, self-made opium, and codeine), V. also abused cannabis and alcohol. He experienced multiple craniocerebral traumas and underwent drug treatment 9 times. In 2006 V. used heroin and codeine.

V. was diagnosed HIV-positive in 2005 (way of transmission—injecting drug use) and currently receives HIV treatment in the Moscow AIDS center. V.'s co-infections include viral hepatitis B and C. His opportunistic infections are pneumocist pneumonia, herpes, candidiasis.

In September 2005 V. started antiretroviral therapy with Stocrin and Combivir because of the progression of HIV disease (CD4-120, viral load—7 200). His average adherence to ARVs in the period January-March 2006 has been 70%. According to V.'s explanations, this low level of adherence is related to his unstable life style and ‘bad state of health’ because of heroin and codeine use and related opiate withdrawal.

In April 2006 V. was administered Nalbuphine, 2 cc IM twice a day that led to discontinuance of the use of heroin and ‘street drugs’ (self-made opium and codeine). His mental state improved and he remains free of heroin and codeine but his problems with alcohol persist. Nevertheless V's average adherence to ART during April has been 95%. No aggravation of opportunistic infections or HCV have been observed.

### Example 6A

### C3-P-Rus

L., was a 31 years old female, not married, unemployed, HIV-infected (symptomatic stage), with heavy opioid dependency.

At the age of 16 L. tried illicit drugs (self-made opium) for the first time, and from the age of 18 regularly used psychoactive drugs. In addition to opiates (self-made opium, heroin, codeine), L. started to use amphetamines in 2005. L. attempted suicide multiple times.

Over the period of her drug use, L. received treatment on several occasions. The maximum duration of the remission has been 2 years, which has coincided with imprisonment. After release from prison, L. restarted to use heroin immediately. The last in-patient drug treatment took place in July-August 2005 with the remission after the hospital of 3.5 weeks. L. has been registered in the Novokuznetsk drug treatment center.

L was diagnosed HIV-positive in 2002 (way of transmission—injecting drug use) and has been receiving HIV treatment in the Russian Federal AIDS center. L's co-infections include HCV infection and syphilis. In 2004-2005 L had a repetitive acute bacterial pneumonia, severe candida infections, toxoplasmosis.

In December 2005 L. started antiretroviral therapy with Efavirenz and Combivir because of the progression of HIV disease (CD4-192). Average adherence to ARVs during the period of January-March 2006 has been 68%.

In April 2006 L. was administered Nalbuphine, 2 cc IM twice a day and discontinued the use of heroin. Her mental state profoundly improved and the drug cravings decreased. No attempts of suicide have been observed and currently L. is trying to obtain employed.

Average adherence to ART during April has been 93%. No aggravation of opportunistic infections or of HCV have been observed.

### Example 7A

### C4-P-Rus

S., female, 38 years old, married, has 1 child, is invalid and HIV-infected (AIDS). S displayed heavy opioid dependency.

From the age of 21 regularly used psychoactive drugs: opiates (self-made opium, heroin, codeine), amphetamines, cannabis, and GBH (gammahydroxybutyrate). During the period of her drug use, S. tried to stop drug use for 4 times. The maximum duration of the remission has been 1.5 years in 1998, other periods of remission didn't last for more than 1 month. The last remission took place in December 2005.

S. was diagnosed HIV-positive in 2000 (way of transmission—injecting drug use). She was placed on HIV treatment in the St. Petersburg AIDS center. Her co-infections included: chronic viral hepatitis B, C and D.

S started antiretroviral therapy with Zidovudine, Lamivudine, and Efavirence and Combivir in March 2005 because of the development of AIDS (CD4-14). ARV had low effectiveness in L and after 12 months of treatment her immune status increased only up to 80.

Her average adherence to ARVs in January-March 2006 has been 98%.

S. complaints on severe chronic pains of unclear etiology in her shins and hips. Since December of 2005 she has been administrated non-steroid pain-killers and tramadol, with low effectiveness.

In April 2006 S. was administered Nalbuphine, 2 cc IM twice a day that led to both pain relief and discontinuance of the use of heroin. S. says that her abstinence of heroin is directly related to the anesthetizing effect of Nalbuphine. Mental state profoundly improved; cravings decreased. Her average adherence to ART during April has been 93%. No aggravation of opportunistic infections or of HCV have been observed.

### Example 8A

A pilot study was initiated to evaluate treatment with Nalbuphine (agonist-antagonist) in the context of HIV prevention, treatment and care interventions. The first pilot study of the effects of Nalbuphine administration to IDU (injectable drug users) started at the 8^{th} of August 2006 in Moscow. It is still being conducted on outpatient basis.

Duration of individual treatment varied from patient to patient (from 10 days to 180 days) depending on patient's condition, life-style, substance abuse history, etc. The frequency of administration varied from 20 mg per patient bid (initially) to 20 mg per patient per week (after 30 days).

The objective of the study is to compare this method with the other methods of drug treatment typical for Russia, and to measure the effects of out-patient Nalbuphine administration using following criteria:

improvement and stability of psychological factors

increase of social activity

changes in risky behavioral practices (both injecting and sexual)

adherence to essential treatment

involvement into HIV programs: HIV antibody testing, diagnostics and treatment

The clinical trial included 19 clients, all with simultaneous codeine addiction, 7 patients living with AIDS, 4 being multiple substance users. In all cases, the patient was a difficult case (5-11 years of heroin abuse). 3 of the patients had very high (3-5 g a day) doses of heroin abuse.

All patients were between 18-50 years old, diagnosed with opiate abuse (in some cases in combination with other substances). They were admitted to the study after three days of opiate abstinence.

Patients with psychiatric, oncological, diabetic conditions, chronic kidney illnesses, and pregnancy were excluded from the study.

18 patients stopped heroin and codeine consumption. All HIV-positive showed higher involvement/adherence to HIV treatments. No side effects or drug-drug interactions were observed. All patients remarked on improved quality of life.

### Example 9A

The results of a study of 28 patients who received Nalbuphine for up to 6 months are as follows.

Significant reduction of total heroin and other opiate consumption (up to 75%) with two thirds of people staying clean (by periodic urine tests for opiates) while on Nalbuphine therapy.

All patients demonstrated clear reduction of cravings for drug. An inverse correlation between administrated dose of Nalbuphine and heroin consumption was found.

There were no observed instances of overdose, side effects or new infections associated with injectable drugs.

Quality of life was improved and patients showed:

a) Healthy weight gains

b) Improvement in night sleep

c) Improvement in relationships (with family, relatives)

d) People found new or better employment, reported increase in productivity.

e) Reduction of criminal behavior (e.g. buying or selling drugs, theft, arrests by police etc.)

All patients with HIV infection have started regular immune status check ups and ARV therapy. Those already receiving ARVT have reported nearly perfect compliance (adherence). Other patients have undergone voluntarily training and testing for HIV.

### B. Formulation

In the formulation, nalbuphine can be identified by methods such as UV-spectrophotometric analysis, liquid column chromatography and methods of planar chromatography.

Quantity determination of nalbuphine in the formulation and also in bioavailability evaluation can be monitored by UV-spectrophotometric analysis or high-performance liquid chromatography (HPLC).

### Example 1B

The following ingredients were used:

1. Nalbuphine hydrochloride—10-30%

2. Carbopole (71 G)—22-28%

3. Aerosile—0.2-3%

4. Microcrystal cellulose—5-20%

5. Magnesium or calcium stearate—0.5-1%

6. Lactose—the rest.

In milligrams, the ingredients were as follows:

1. Nalbuphine hydrochloride—15-150 mg

2. Carbopole (71 G)—33-125 mg

3. Aerosile—0.3-13.5 mg

4. Microcrystal cellulose—7.5-90 mg

5. Magnesium or calcium stearate—0.8-4.5 mg

6. Lactose—the rest.

The determined quantity of the components 1 and 3 were mixed, then components 2, 4 and 6 were added. After the mixing component 5 was added. The resulting mixture was processed into a tablet.

The study of nalbuphine release kinetics was done in the conditions corresponding to the requirements of General Pharmacopoeia Article 42-0003-04 of Ministry of Health of the Russian Federation using an apparatus like a rotating basket (referred to herein as apparatus 1), as specified in the Article 42-0003-04 or an impeller mixer (referred to herein as apparatus 2), as specified in the Article 42-0003-04. Water or buffer solutions with pH 6.5-7.6 were used as the dissolution medium. The dissolution medium is 900 ml. One tablet, of up to 150 mg) was placed in the dissolution medium (in the glass with the medium or in the basket). The time of the experiment was 8 hours and six parallel determinations were carried out for every medium.

At the end of the test, a sample was taken and the quantity of nalbuphine in the sample was analyzed and compared with the initial quantity used in the tablet, to determine how much passed into the solution during the stated period of time. Min, max and medium values were derived from the six results obtained.

It was determined that the use of apparatus 2 resulted in the tablet adherence to the bottom of the glass, and apparatus 2 was deemed unsuitable for these tests. Further experiments were conducted using apparatus 1.

The results are shown in Table 1B, below:

TABLE 1B

| | Quantity of nalbuphine passing into the solution, % | | | | | | | | | | | |
|---|---|---|---|---|---|---|---|---|---|---|---|---|
| | 1 hour | | | 3 hours | | | 5 hours | | | 8 hours | | |
| Medium | Min | Max | Ave | Min | Max | Ave | Min | Max | Ave | Min | Max | Ave |
| Water | 12.42 | 16.94 | 14.12 | 37.43 | 42.45 | 39.91 | 67.84 | 79.08 | 71.62 | 94.01 | 98.23 | 96.43 |
| Buffer | 11.98 | 17.21 | 15.46 | 34.38 | 42.11 | 38.27 | 67.94 | 77.54 | 69.19 | 92.44 | 98.64 | 94.67 |
| pH 6.5 | | | | | | | | | | | | |
| Buffer | 12.06 | 19.64 | 15.93 | 33.21 | 40.67 | 36.64 | 66.34 | 79.99 | 74.33 | 89.90 | 98.75 | 93.97 |
| pH 6.8 | | | | | | | | | | | | |
| Buffer | 11.82 | 18.22 | 14.78 | 36.09 | 42.84 | 38.71 | 59.87 | 80.67 | 70.72 | 90.03 | 97.44 | 94.64 |
| pH 7.6 | | | | | | | | | | | | |

Analysis of the results indicate that release profiles for different media do not have a statistically significant differences and that prolongation is achieved during 8 hours.

Basing on the data of the Table 1B, further kinetics study was done in buffer medium with pH 6.8 in order to provide conformity with physiological medium and to stabilize the conditions of the trial.

### Example 2B

All the ingredients were the same, as in Example 1B except that carbopole 71 G was replaced with hydroxypropylcellulose which was used in the same proportions. The prolongation data are given in Table 2B.

TABLE 2B

| | Quantity of nalbuphine passing into the solution, % | | | | | | | | | | | |
|---|---|---|---|---|---|---|---|---|---|---|---|---|
| | 1 hour | | | 3 hours | | | 5 hours | | | 8 hours | | |
| Medium | Min | Max | Average | Min | Max | Average | Min | Max | Average | Min | Max | Average |
| Buffer | 9.81 | 19.39 | 14.67 | 36.78 | 39.75 | 38.77 | 64.31 | 75.28 | 71.98 | 84.12 | 95.62 | 91.14 |
| pH 6.5 | | | | | | | | | | | | |

As seen from Table 2B, hydroxypropylcellulose as well as carbopole provided prolonged release of nalbuphine from the matrix during 8 hours. Release curves for these both ingredients are similar. In case with HPC there is a data scattering in the stated time period, what may be related to the high strength of tablets produced with HPC.

### Example 3B

The experiment was conducted as described in Examples 1B and 2B, above. In addition, the tablet was covered with a gastro-resistant coating which was produced from acetylphthalylcellulose.

Furthermore, before the study of release kinetics, an acidic stage was introduced in the buffer solution of pH 6.8. Specifically, the tablet integrity was studied at a 0.1 M solution of hydrochloric acid, imitating digestive juice, under the same conditions for two hours. It was desired that the quantity of nalbuphine passing into the solution would not increase by more than 10%.

The results are shown in Table 3B, below:

TABLE 3B

| | Nalbuphine release, % | | | | |
|---|---|---|---|---|---|
| | | Phosphate buffer pH 6.8 | | | |
| Modificator | | 1 | 3 | 5 | 8 |
| Of release | 0.01M solution HCl, 2 hours | hour | hours | hours | hours |
| Carbopole | 0.09%, film without damages | 15.78 | 42.44 | 73.19 | 97.46 |
| HPMC | 0.11%, film without damages | 16.57 | 40.64 | 74.44 | 98.11 |

The data in Table 3B show that the use of acetylphtalylcellulose as a film-forming ingredient inhibits the influence of acidic medium on tablet content. Integrity of tablet ingredients and in particular of nalbuphine chloride is enhanced when exposed to a medium that mimics the digestive juice. The prolongation in the buffer system remains approximately without changes in comparison with Examples 1B and 2B.

### Example 4B

The experiments were conducted as described in Example 3B, except that the tablet was covered with an intestinal-soluble coating, specifically Acryl-eze® system (Colorcon, Great Britain). Results are shown in Table 4B, below:

TABLE 4B

| | Nalbuphine release, % | | | | |
|---|---|---|---|---|---|
| | | Phosphate buffer pH 6.8 | | | |
| Modificator | | 1 | 3 | 5 | 8 |
| of release | 0.01M solution HCl, 2 hours | hour | hours | hours | hours |
| Carbopole | 0.07%, film without damages | 14.98 | 39.70 | 74.62 | 98.49 |
| NPMC | 0.10%, film without damages | 18.01 | 41.45 | 76.89 | 97.53 |

The data show that the use of Acryl-eze® system as a film-forming ingredient inhibited the influence of the acidic medium on the nucleus content, increasing resistance to medium that mimics the digestive juice and preventing nalbuphine destruction under its influence. The prolongation in the buffer system remained approximately without changes in comparison with Examples 1B and 2B.

### Example 5B

The following ingredients were employed:

1. Nalbuphine hydrochloride—32-55%

2. Mixture of carbopole+HPMC—25-35%

3. Aerosile—0.2-3%

4. Microcrystal cellulose—5-20%

5. Magnesium or calcium stearate—0.5-1%

6. Lactose—the rest.

7. Film-forming Acryl-eze® system—1-5% from the tablet system.

Carbopole 71 G—HPMC mixtures were used in proportion of 0 to 100% with respect to HPMC.

Experimental data of kinetics study are given in Table 5B below:

TABLE 5B

| | Time for achieving the level of | | |
|---|---|---|---|
| | nalbuphine release | | |
| | in phosphate buffer | | |
| | pH 6.8 after acidic stage, hours | | |
| Correlation | From 10 | From | From 80% |
| carbopole/HPMC | 50 to 25% | to 75% | and more |
| 0/1 | 1.5-2.5 | 9-15 | From 17 |
| 1/3 | 1.5-2 | 9-16 | from 19 |
| 1/1 | 1.5-2.5 | 9-17 | From 19 |
| 3/1 | 1.5-2.5 | 9-18 | From 21 |
| 1/0 | 1.5-2 | 9-18 | from 23 |

Depending on correlation of the prolongating ingredients, prolongation is achieved during 15-23 hours, what allows modifying the intake of the medicine in accordance with different treatment regimens, e.g., scheduling, in different groups of patients.

### Example 6B

This example was carried out as described in Example 5B except that the quantity of carbopole/HPMC mixture was decreased to 10-20% from the composition mass. The results are shown in Table 6B below:

TABLE 6B

| | Time for achieving the level of | | |
|---|---|---|---|
| | nalbuphine release in phosphate | | |
| | buffer pH 6.8 after acidic stage, hours | | |
| Correlation | from 10 | from | from |
| carbopole/HPMC | to 25% | 50 to 75% | 80% and more |
| 0/1 | 0.5-1.5 | 3-5 | From 7 |
| 1/3 | 0.5-1.5 | 3-5 | From 7 |
| 1/1 | 0.5-1.5 | 3-6 | From 7 |
| 3/1 | 0.5-1.5 | 4-6 | From 8 |
| 1/0 | 0.5-1.5 | 4-6.5 | From 8 |

### Example 7B

The experiment was conducted as in Examples 5B and 6B, but carbopole was excluded and only HPMC was used as a release-modifying agent. Dry mixing was replaced by moist granulation with the use of povidone solution 5-15% (the quantity of povidone in the composition was from 1 to 5%), with further application of lubricants, compounding into a tablet and applying of one of the gastric-resistant coverings.

The results of the kinetics study are given in Table 7B.

TABLE 7B

| | Nalbuphine release, % | | | | |
|---|---|---|---|---|---|
| Quantity | | Phosphate buffer pH 6.8 | | | |
| of HPMC, % | 0.01M solution HCl, 2 hours | 1 h | 5 h | 8 h | 24 h |
| 10 | 0.10%, film without damages | 14.47 | 60.13 | 98.71 | — |
| 20 | 0.10%, film without damages | 12.98 | 51.02 | 84.97 | 99.06 |
| 40 | 0.10%, film without damages | 10.11 | 52.34 | 79.99 | 98.37 |

Depending on correlation of the prolonged release, prolonged release is achieved during 8-24 hours, allowing modification of intake of the medicine in accordance with different treatment schedules as desired for different groups of patients.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for stabilizing inconsistencies associated with opioid dependence, the method comprising:
a. selecting a patient addicted to an opioid; and
b. administering to the patient an opioid agonist-antagonist for at least 14 days, thereby stabilizing said inconsistencies.

2. The method of claim 1, wherein the opioid is selected from the group consisting of heroin, codeine, codeine phosphate, tramadol, home-made opiate and any combination thereof.

3. The method of claim 1, wherein the opioid agonist-antagonist is selected from the group consisting of nalbuphine, butorphanol, pentazocine and any combination thereof.

4. The method of claim 1, wherein the opioid agonist-antagonist is nalbuphine.

5. The method of claim 1, wherein the patient also suffers from an infection selected from the group consisting of HIV, tuberculosis, herpes, hepatitis B, hepatitis C and any combination thereof.

6. The method of claim 5, wherein the patient also receives ARV treatment.

7. The method of claim 1, wherein the agonist-antagonist is administered by injection.

8. The method of claim 1, wherein the agonist-antagonist is administered orally.

9. The method of claim 1, wherein the agonist-antagonist is administered once or twice daily.

10. The method of claim 1, wherein the agonist-antagonist is a synthetic, semi-synthetic or naturally occurring compound.

11. The method of claim 1, wherein the patient also has an alcohol dependency.

12. The method of claim 1, wherein said inconsistencies are behavioral, psychological or both.

13. The method of claim 1, wherein the opioid agonist-antagonist is administered in combination with one or more agonist(s) and/or antagonist(s).

14. The method of claim 1, wherein the opioid agonist-antagonist is administered in combination with another treatment for opiate addiction.

15. A method for maintaining an opioid-dependent patient on a treatment regimen, the method comprising:
a. selecting an opioid-dependent patient in need of an average adherence to the treatment regimen of at least 95 percent; and
b. administering to the selected patient a combination of a treatment component and, for at least a month, an opioid receptor agonist-antagonist, whereby the patient's average adherence to the treatment regimen, at he end of he at least a month, is no less than 95 percent.

16. The method of claim 15, wherein the treatment regimen is an ARV regimen.

17. The method of claim 16, wherein the ARV regimen includes abacavir, didanosine, emtricitabine, lamivudine, zidovudine, stavudine, stavudine, tenofovir, zalcitabine, delavirdine, efavirenz, nevirapine, amprenavir, atazanavir, fosamprenavir, indinavir, lopinavir, ritonavir, nelfinavir, saquinavir, enfuvirtide, acyclovir, interferons and any combination thereof.

18. The method of claim 15, wherein the patient's average adherence, at he end of the at least a month is at least.

19. The method of claim 15, wherein the patient is HIV-positive.

20. The method of claim 15, wherein the opioid is selected from the group consisting of heroin, codeine, codeine phosphate, tramadol, home-made opiate and any combination thereof.

21. The method of claim 15, wherein the opioid agonist-antagonist is selected from the group consisting of nalbuphine, butorphanol, pentazocine and any combination thereof.

22. The method of claim 15, wherein the agonist-antagonist is a synthetic, semi-synthetic or naturally occurring compound.

23. The method of claim 15, wherein the patient also has an alcohol dependency.

24. The method of claim 15, wherein the treatment component and the opioid agonist-antagonist are administered on an individualized schedule.

25. The method of claim 15, wherein the treatment component and the opioid agonist-antagonist are administered on the same schedule.

26. The method of claim 15, wherein the opioid agonist-antagonist is administered in combination with one or more agonist(s) and/or antagonist(s).

27. The method of claim 15, wherein the opioid agonist-antagonist is administered in combination with another treatment for opiate addiction.

28. A method for maintaining an HIV-infected, opioid-dependent patient on an ARV regimen, the method comprising administering to the patient an ARV component in combination with an opioid receptor agonist-antagonist, wherein the opioid receptor agonist antagonist is administered for no less than a month and, at the end of the at least a month, the patient's average adherence to the ARV regimen is at least 95 percent.

29. A method for treating an HIV-infected patient, the method comprising:
a. selecting an opioid-dependent patient having an average adherence to an ARV regimen that is less that 95 percent; and
b. administering to the patient ARV medication in combination with nalbuphine.

30. A method for maintaining an average compliance to an ARV regiment of at least 95 percent in an opioid-dependent patient, the method comprising administering to the patient ARV medication in combination with an opioid receptor agonist-antagonist.

31. A non-injectable formulation for managing opioid-addiction, the formulation comprising:
a. an active component including free-base nalbuphine or pharmaceutically acceptable derivative or salt of nalbuphine; and
b. a release modifying component,
wherein the formulation is in tablet or capsule form.

32. The formulation of claim 31, wherein the free-base nalbuphine or pharmaceutically acceptable derivative or salt of nalbuphine is present in the formulation in an amount of at least 5 milligrams.

33. The formulation of claim 31, wherein the release modifying component is a carbomer, hydroxypropylmethylcellulose or any combination thereof.

34. The formulation of claim 31, further comprising an additive component.

35. A kit comprising the formulation of claim 31.

36. A transdermal delivery device for managing opioid-addiction, wherein said device includes an opioid agonist-antagonist.

37. A non-injectable formulation for managing opioid-addiction, the formulation comprising an agonist-antagonist, wherein the formulation is in the form of a syrup, suppository, powder, inhalable aerosol, sublingual spray, intranasal spray or intranasal aerosol.

38. A method for severing an association with injectable opiates, in an opiate using patient, the method comprising administering to the opiate using patient a non-injectable formulation which includes an agonist-antagonist for at least 14 days.

39. Use of an agonist-antagonist for the manufacture of a medicament for stabilizing inconsistencies associated with opioid dependence.

40. Use of an agonist-antagonist for the manufacture of a medicament for maintaining an opioid-dependent patient on a treatment regimen.

41. The method of claim 40, wherein the patient is HIV-positive and the treatment regimen is an ARV regimen.
